(19) United States
(12) Patent Application Publication
(10) Pub. No.: US 2023/0012812 A1
(43) Pub. Date: Jan. 19, 2023

(54) MEDICAL INSTRUMENT SET, DELIVERY SYSTEM, AND EMBOLIC DEVICE DELIVERY MEDICAL SYSTEM

(71) Applicant: TERUMO KABUSHIKI KAISHA, Tokyo (JP)

(72) Inventors: Hideaki SHIBATA, Hadano-shi (JP); Yuki ITOH, Hadano-shi (JP); Eri IKUNO, Hadano-shi (JP)

(73) Assignee: TERUMO KABUSHIKI KAISHA, Tokyo (JP)

(21) Appl. No.: 17/954,384

(22) Filed: Sep. 28, 2022

Related U.S. Application Data
(63) Continuation of application No. PCT/JP2021/009409, filed on Mar. 10, 2021.

(30) Foreign Application Priority Data
Mar. 31, 2020 (JP) 2020-062217

(51) Int. Cl. A61B 17/12

(52) U.S. Cl. CPC A61B 17/12113; A61B 2017/1205; A61B 2017/306

(57) ABSTRACT

To permit placement of an embolic device embolic device at a desired indwelling position in an aneurysm and readjustment of the indwelling position of the embolic device that is already indwelled, a medical instrument set includes an embolic device insertable into a delivery catheter, and configured to, when the embolic device is indwelled in an aneurysm, absorb a liquid in the aneurysm and expand, and an elongated delivery pusher insertable into the delivery catheter and configured to push the embolic device out of the delivery catheter into the aneurysm. The delivery pusher includes an elongated main body and an aspiration lumen that is formed to penetrate to main body in an axial direction from a distal end to a proximal end of the main body and causes the delivery pusher to aspirate and hold the embolic device.

### CROSS-REFERENCES TO RELATED APPLICATIONS

This application is a continuation of International Patent Application No. PCT/JP2021/009409 filed on Mar. 10, 2021, which claims priority to Japanese Patent Application No. 2020-062217 filed on Mar. 31, 2020, the entire content of both of which is incorporated herein by reference.

### TECHNOLOGICAL FIELD

The present invention generally relates to a medical instrument set, a delivery system including the medical instrument set, and an embolic device delivery medical system including the delivery system.

### BACKGROUND DISCUSSION

There is no drug treatment to prevent an aneurysm (aortic aneurysm) generated in an aorta of a patient from increasing in diameter and rupture, and surgical treatment (surgery) is generally performed for the aneurysm with a risk of rupture. Further, in a surgery of the aortic aneurysm of the related art, artificial blood vessel substitution surgery in which an artificial blood vessel (stent graft) is transplanted by laparotomy or thoracotomy has been a mainstream, and in recent years, application of lower invasive stent graft interpolation (endovascular aneurysm repair: EVAR) has been rapidly expanding.

For example, in stent graft interpolation for an abdominal aortic aneurysm (AAA), a catheter having a stent graft attached to a distal end thereof is inserted from a peripheral blood vessel of the patient, and the stent graft is opened and indwelled in an aneurysm target lesion, thereby blocking a blood flow to the aneurysm and preventing the aneurysm from rupturing.

In general, a stent graft used in the stent graft interpolation has a structure in which two types of members, that is, a “main body portion” including bifurcated portions bifurcated into a substantially Y-shape and “limb portions” attached to the bifurcated portions and attached to a right iliac artery and a left iliac artery, respectively, are assembled.

Therefore, in the stent graft interpolation, so-called “endoleak” in which the blood flow remains in the aneurysm may occur due to blood leakage from a periphery of the stent graft due to insufficient adhesion of the interpolated stent graft, backflow of blood from a thin blood vessel (side branch blood vessel) branched from the aneurysm, or the like. In this case, since a pressure is applied to a wall of the aneurysm by the blood flow entering the aneurysm, there is a potential risk of rupture of the aneurysm.

U.S. Pat. No. 9,561,096 discloses a device including a catheter capable of holding a compressed relatively elongated sponge (embolic device) in a lumen of the catheter and a plunger for pushing the embolic device held in the catheter into an aneurysm filled with blood in order to block blood flow remaining in an aortic aneurysm due to endoleak. Since the sponge used in this device expands immediately when exposed to the blood, the sponge is pushed into the aneurysm and expands when absorbing the blood in the aneurysm, and the sponge is indwelled in the aneurysm in this state to block the blood flow and prevent rupture.

In endoleak embolization performed in U.S. Pat. No. 9,561,096, when the embolic device to be used enters slight gaps that can be generated at connection portions between bifurcated portions and limb portions after a stent graft is indwelled, the connection portions may be expanded when the embolic device is expanded to cause the endoleak.

Further, in order to block the blood flow from a side branch blood vessel branched from the aneurysm, the embolic device is indwelled in the vicinity of the side branch blood vessel, and when the embolic device enters from a communication port between a wall portion of the aneurysm and the side branch blood vessel, there is a concern that distal occlusion may occur due to the embolic device flowing through the blood vessel.

As described above, a surgeon needs to pay attention to indwell the embolic device so as not to cause the endoleak or other problems caused by the embolic device to be indwelled in the endoleak embolization.

However, in the device disclosed in U.S. Pat. No. 9,561,096, the embolic device can be pushed out into the aneurysm and indwelled, but the pushed embolic device cannot be pulled back. Therefore, once the embolic device is pushed into the aneurysm, it is extremely difficult to readjust an indwelling position of the embolic device.

### SUMMARY

The medical instrument set, delivery system, and embolic device delivery medical system disclosed here are configured to indwell an embolic device at a desired indwelling position in an aneurysm and readjust the dwelling position of the indwelled embolic device.

A medical instrument set according to one embodiment disclosed by way of example includes: an embolic device insertable into a delivery catheter and configured to, when the embolic device is indwelled in an aneurysm, absorb a liquid in the aneurysm and expand; and an elongated delivery pusher insertable into the delivery catheter and configured to push the embolic device out of the delivery catheter into the aneurysm. The delivery pusher includes an elongated main body and an aspiration lumen that is formed to penetrate the main body in an axial direction from a distal end to a proximal end of the main body and causes the pusher to aspirate and hold the embolic device.

A delivery system according to the present embodiment includes the medical instrument set and an aspiration and ejection unit configured to aspirate or eject a fluid from or to the aspiration lumen.

An embolic device delivery medical system according to an embodiment disclosed by way of example includes: a tubular delivery catheter that includes a distal portion terminating at a distal end, with the tubular delivery catheter also including a proximal end and a lumen extending between the distal end and the proximal end of the tubular delivery catheter, and the tubular delivery catheter being configured to have its distal portion introduced into a living body and positioned in an aneurysm in the living body; and an embolic device-filled catheter set that includes an embolic device in combination with an embolic device catheter, wherein the embolic device catheter including a distal end, a proximal end and a lumen extending between the distal end and the proximal end, and the embolic device being positioned in the lumen of the embolic device catheter such that the embolic device is removable from the lumen of the embolic device catheter and is indwellable in an aneurysm. The embolic device is configured to absorb liquid in the aneurysm when the embolic device is indwelled in the aneurysm and to expand upon absorbing the liquid, with the embolic device catheter being configured to be inserted into the lumen in the tubular delivery catheter when the embolic device is positioned in the lumen in the embolic device catheter. A delivery pusher includes an elongated main body and an aspiration lumen, with the elongated main body of the delivery pusher being insertable into the delivery catheter and configured to push the embolic device out of the delivery catheter when the distal portion of the tubular delivery catheter is positioned in the aneurysm in the living body so that the embolic device is located in the aneurysm. The aspiration lumen extends through the elongated main body of the delivery pusher in an axial direction from a distal end of the elongated main body to a proximal end of the elongated main body to permit aspiration to be applied to the embolic device when the embolic device is located at the distal end of the elongated main body to hold the embolic device at the distal end of the elongated main body of the delivery pusher.

According to another aspect, a method comprises: introducing a distal portion of a delivery catheter into a living body and positioning the distal end of the delivery catheter in an aneurysm in the living body, wherein the delivery catheter includes a distal end, a proximal end and a lumen extending throughout the delivery catheter from the distal end of the delivery catheter to the proximal end of the delivery catheter; and introducing a distal end of a delivery pusher into the delivery catheter by way of the proximal end of the delivery catheter, wherein the introducing of the distal end of the delivery pusher into delivery catheter by way of the proximal end of the delivery catheter includes introducing the distal end of the delivery pusher into the delivery catheter while an embolic device is located inside the delivery catheter. The method additionally includes moving the delivery pusher in a forward direction to push the embolic device out of the delivery catheter so that the embolic device is located outside the delivery catheter and in the aneurysm, applying aspiration to the lumen in the delivery pusher to hold the embolic device that is located in the aneurysm against a distal end of the delivery pusher, moving the embolic device in the aneurysm while the embolic device is held against the distal end of the delivery pusher by the aspiration; stopping the applying of the aspiration to the lumen in the delivery pusher to release the embolic device in the aneurysm; and moving the delivery pusher in a rearward direction while the embolic device remains in the aneurysm.

According to at least one embodiment of the invention, the embolic device can be indwelled at a desired indwelling position in the aneurysm and the indwelling position of the embolic device that is already indwelled can be readjusted, and thus a problem including occurrence of endoleak caused by the embolic device can be prevented.

### BRIEF DESCRIPTION OF THE DRAWINGS

FIG. 1 is a diagram showing a configuration of a medical instrument set and a configuration of a delivery system according to the present embodiment.

FIG. 2 is a diagram showing a configuration of an embolic device delivery medical system according to the present embodiment.

FIG. 3A is a perspective view schematically showing a delivery pusher and an embolic device that are accommodated in a delivery catheter.

FIG. 3B is an end view of the delivery pusher and the embolic device as viewed from a distal end side.

FIG. 4A is a view showing a usage example of the embolic device delivery medical system according to the present embodiment and showing a state before the embolic device is pushed out by the delivery pusher.

FIG. 4B is a view showing a usage example of the embolic device delivery medical system according to the present embodiment and showing a state in which the embolic device is being pushed out by the delivery pusher.

FIG. 4C is a view showing a usage example of the embolic device delivery medical system according to the present embodiment and showing a state in which the embolic device is pulled back by the delivery pusher.

FIG. 4D is a view showing a usage example of the embolic device delivery medical system according to the present embodiment and showing a state in which the embolic device is pushed out while being aspirated by the delivery pusher.

FIG. 5A is a diagram showing an operation example of the embolic device delivery medical system according to the present embodiment and showing a state in which the delivery catheter is delivered into an aneurysm.

FIG. 5B is a diagram showing an operation example of the embolic device delivery medical system according to the present embodiment and showing a state in which a stent graft is developed in the aneurysm.

FIG. 5C is a diagram showing an operation example of the embolic device delivery medical system according to the present embodiment and showing a state in which the embolic device is pushed out of the delivery catheter.

FIG. 5D is a diagram showing an operation example of the embolic device delivery medical system according to the present embodiment and showing a state in which the embolic device is indwelled in the aneurysm.

FIG. 5E is a diagram showing an operation example of the embolic device delivery medical system according to the present embodiment and showing a state in which the embolic device absorbs a liquid in the aneurysm and swells.

FIG. 6 is a perspective view showing a delivery pusher of a medical instrument set according to a modification (first modification) of the present embodiment.

FIG. 7 is a perspective view showing a delivery pusher of a medical instrument set according to another modification (second modification) of the present embodiment.

FIG. 8 is a perspective view showing a delivery pusher of a medical instrument set according to another modification (third modification) of the present embodiment.

### DETAILED DESCRIPTION

Hereinafter, an embodiment of the medical instrument set, delivery system, and embolic device delivery medical system will be described in detail with reference to the drawings. The embodiment shown here is an example for embodying a technical idea of the medical instrument set, delivery system, and embolic device delivery medical system disclosed here, and does not limit the invention. Further, other aspects, examples, operational techniques, and the like that can be implemented by those skilled in the art without departing from the gist and spirit of the disclosure here and all are included in the scope and the gist of the invention, and are included in the invention described in the claims and the scope of equivalents thereof.

Further, in the accompanying drawings, for convenience of illustration and understanding, a scale, an aspect ratio, a shape, and the like may be changed from actual ones and may be schematically expressed as appropriate, and the drawings are just examples and do not limit the interpretation of the invention.

A medical instrument set 100, a delivery system 200, and an embolic device delivery medical system 300 according to the present embodiment are intended to aspirate and hold an embolic device 10 by aspirating a fluid (gas or liquid) for holding the embolic device 10 from an aspiration lumen 22 provided in a delivery pusher 20 when the embolic device 10 is to be indwelled in an aneurysm (for example, an aneurysm) in a blood vessel, thereby making it possible to adjust an indwelling position of the embolic device 10 in the aneurysm.

The medical instrument set 100, the delivery system 200, and the embolic device delivery medical system 300 according to the present embodiment may be applied to endoleak embolization for stent graft interpolation of an abdominal aortic aneurysm (AAA), which is an example of a treatment method for preventing rupture of the aneurysm generated in the blood vessel. In addition, the treatment method in which the medical instrument set 100, the delivery system 200, and the embolic device delivery medical system 300 according to the present embodiment are used is not limited to the endoleak embolization for the stent graft interpolation of the abdominal aortic aneurysm, and is also applicable to other intervention treatment methods for preventing the rupture of the aneurysm generated in the blood vessel.

In the present specification, in an operation direction of each part constituting the embolic device delivery medical system 300, for example, a side on which the embolic device 10 is to be conveyed into the aneurysm in a direction along an axial direction of a delivery catheter 40 is referred to as a “distal end side”, and a side that is located on a side opposite to the distal end side and on which a surgeon performs an operation with his/her hands (a side from which the delivery catheter 40 is removed) is referred to as a “proximal end side”. In addition, a direction parallel to the axial direction of the delivery catheter 40 and in which the delivery pusher 20 advances (is inserted) or retreats (is removed) is referred to as an “advancing and retreating direction”, and a direction along a circumferential direction (a direction around the axial direction of the delivery catheter 40) of the delivery catheter 40 is referred to as a “rotation direction”.

[Configuration]

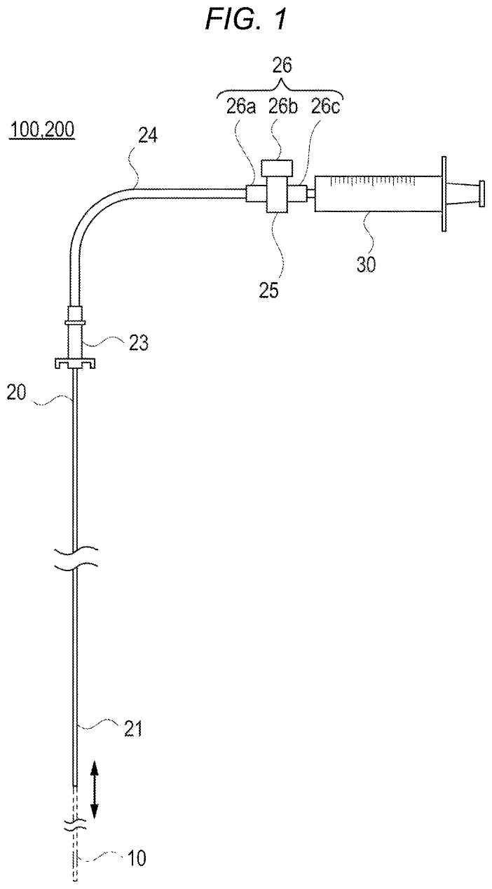

First, configurations of the medical instrument set 100, the delivery system 200, and the embolic device delivery medical system 300 according to the present embodiment will be described with reference to FIGS. 1 to 3B.

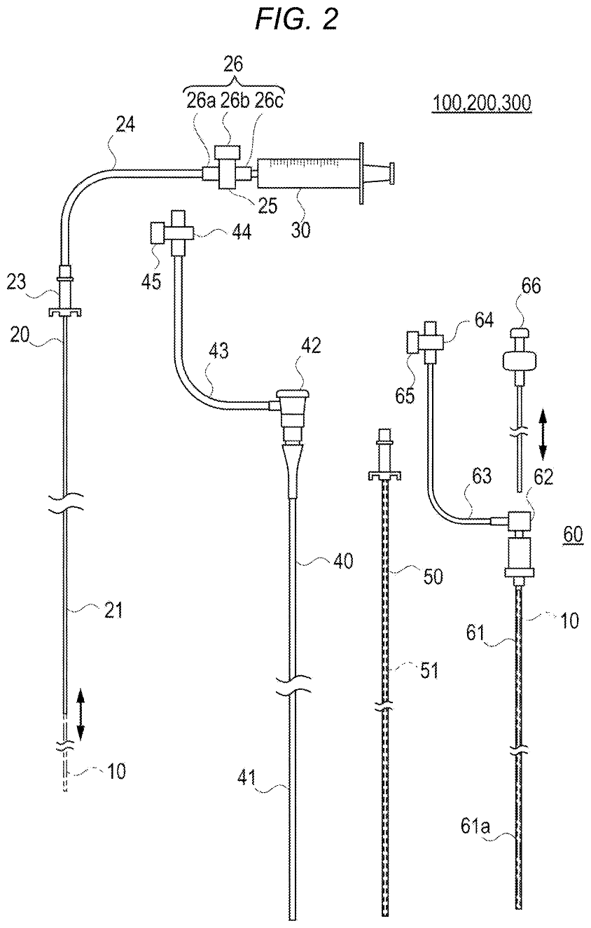

FIG. 1 shows each part constituting the medical instrument set 100 and the delivery system 200 according to the present embodiment, and FIG. 2 shows each part constituting the embolic device delivery medical system 300 according to the present embodiment.

<Medical Instrument Set>

The configuration of the medical instrument set 100 according to the present embodiment will be described. As shown in FIG. 1, the medical instrument set 100 according to the present embodiment includes the embolic device 10 to be indwelled in the aneurysm, and the delivery pusher 20 capable of conveying the embolic device 10 to the indwelling position in the aneurysm while holding the embolic device 10.

[Embolic Device]

The embolic device 10 is indwelled in the aneurysm and expands by absorbing the liquid containing blood flowing into the aneurysm. After being loaded into the delivery catheter 40, the embolic device 10 is conveyed into the aneurysm by the delivery pusher 20.

The embolic device 10 is an elongated fibrous linear body made of an expandable material (polymer material (water-absorbing gel material) or the like) that expands by contacting with an aqueous liquid containing the blood under a physiological condition. In the present embodiment, the embolic device 10 is an elongated member having a substantially circular cross-sectional shape in a direction orthogonal to a longitudinal direction. The cross-sectional shape of the embolic device 10 is not particularly limited, and may be a polygonal shape in addition to the substantially circular shape as in the present embodiment.

Here, the “physiological condition” means a condition having at least one environmental characteristic in a body or a body surface of a mammal (for example, human). Such a characteristic include an isotonic environment, a pH buffering environment, an aqueous environment, a pH near neutral (about 7), or a combination thereof. The “aqueous liquid” includes, for example, body fluids of a mammal (for example, humans) such as isotonic fluid, water, blood, spinal fluid, plasma, serum, glass body fluid, and urine. A size (total length and width dimension) of the embolic device 10 is not particularly limited, and can be appropriately set according to a size of the aneurysm to which the embolic device 10 is to be indwelled, a thickness of a side branch blood vessel such as inferior mesenteric artery or or a lumbar artery interlocked to the aneurysm, and the like.

A constituent material from which the embolic device 10 may be fabricated is not particularly limited as long as it is a material that expands by absorbing at least a liquid such as blood, and has no (or extremely low) harmful effect on a human body even in a state of being indwelled in the aneurysm. In addition, the embolic device 10 may be added or fabricated with a visualization material whose existing position in a living body can be confirmed by a confirmation method such as an X-ray, a fluorescent X-ray, an ultrasound, a fluorescence method, an infrared ray, or an ultraviolet ray.

[Delivery Pusher]

The delivery pusher 20 is operated when the embolic device 10 loaded in a lumen of the delivery catheter 40 is to be conveyed into the aneurysm. A main body 21 of the delivery pusher 20 is a hollow tubular member formed with an aspiration lumen 22 for aspirating the fluid for aspirating, holding, and conveying the embolic device 10 loaded in the delivery catheter 40. The delivery pusher 20 is operated in a predetermined manner in the advancing and retreating direction and the rotation direction by the surgeon, and indwells the embolic device 10 at a desired indwelling position in the aneurysm.

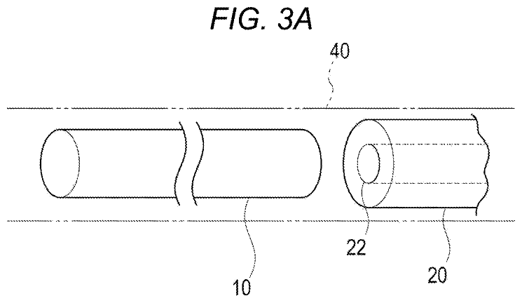

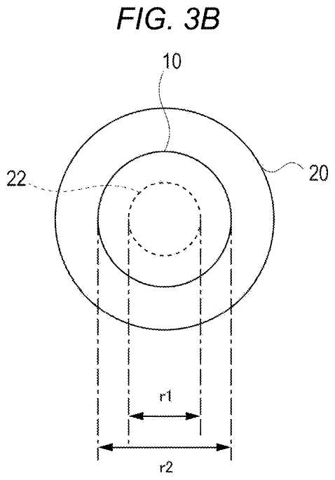

The aspiration lumen 22 is a hole that communicates from the distal end side of the delivery pusher 20 to the proximal end side of the delivery pusher 20 along the axial direction of the main body 21. In order to prevent the embolic device 10 from being suctioned into the aspiration lumen 22, a hole diameter (opening dimension) r1 of the aspiration lumen 22 is set to be shorter or smaller than a width dimension r2 of the embolic device 10 (a length in a lateral direction orthogonal to the longitudinal direction (axial direction) of the embolic device 10). As shown in FIG. 3A or 3B, the embolic device 10 according to the present embodiment has a substantially circular cross-sectional shape orthogonal to the longitudinal direction, and the hole diameter r1 of the aspiration lumen 22 is set to be shorter or smaller than the width dimension (diameter) r2 of the embolic device 10 (r1<r2).

A constituent material from which the main body 21 may be fabricated is not particularly limited as long as it is a material having appropriate hardness and flexibility such that the embolic device 10 can be conveyed. As examples of the constituent material of the main body 21, a resin material such as a polymer material including polyolefin (for example, polyethylene, polypropylene, polybutene, an ethylene-propylene copolymer, an ethylene-vinyl acetate copolymer, an ionomer, or a mixture of two or more types thereof), a polyolefin elastomer, crosslinked polyolefin, polyvinyl chloride, polyamide, a polyamide elastomer, polyester, polyester elastomer, polyurethane, polyurethane elastomer, a fluorous resin, polycarbonate, polystyrene, polyacetal, polyimide, polyetherimide, and aromatic polyether ketone, or a mixture thereof, and a metal material such as a shape memory alloy, stainless steel, tantalum, titanium, platinum, gold, or tungsten can be suitably used.

The delivery pusher 20 includes a hub 23 interlocked to a proximal end side of the main body 21, and a flexible tube 24 having one end connected to a proximal end side of the hub 23 and the other end connected to a three-way stopcock 25.

The hub 23 is an intermediate member that includes a lumen that allows the main body 21 and the tube 24 to communicate with each other, and allows a fluid (liquid, gas, or the like) flowing in from and out to the three-way stopcock 25 to flow through the main body 21 via the tube 24.

A constituent material from which the hub 23 may be fabricated is not particularly limited as long as it is a hard material such as a hard resin. As examples of the constituent material of the hub 23, polyolefin such as polyethylene or polypropylene, polyamide, polycarbonate, polystyrene, or the like can be suitably used.

The one end of the tube 24 is interlocked to the proximal end side of the hub 23, and the other end is connected to a port 26 of the three-way stopcock 25. The tube 24 is a conduit through which a fluid (liquid, gas) flowing in from and out to a priming syringe (not shown) interlocked to the three-way stopcock 25 or an aspiration and ejection unit 30 flows.

The material forming the tube 24 is not particularly limited as long as it is a resin material having a certain degree of flexibility. As examples of a constituent material of the tube 24, polyolefin such as polyethylene, polypropylene, and ethylene-propylene copolymer, polyester such as polyethylene terephthalate, polystyrene, polyvinyl chloride, and the like can be suitably used.

The three-way stopcock 25 communicates with the lumen of the hub 23 and the aspiration lumen 22 of the main body 21 via the tube 24. Three ports 26, which are connection portions of the three-way stopcock 25, are provided, and a proximal end side of the tube 24 is connected to a first port 26a. In addition to a priming syringe for priming the aspiration lumen 22 of the main body 21 with a liquid such as physiological saline, a liquid injection syringe (not shown) for injecting a contrast agent, a drug, or the like into the aspiration lumen 22 may be connected to a second port 26b. Further, a third port 26c is connected to a fluid flow port of the aspiration and ejection unit 30, and allows the fluid (gas or liquid) aspirated or ejected by the aspiration and ejection unit 30 to flow through the aspiration lumen 22 of the main body 21.

As described above, the medical instrument set 100 according to the present embodiment includes the embolic device 10 that is to be indwelled in the aneurysm and expands by absorbing the liquid in the aneurysm, and the delivery pusher 20 that conveys the embolic device 10 into the aneurysm, and the aspiration lumen 22 for aspirating and holding the embolic device 10 is formed in the delivery pusher 20. Therefore, for example, the embolic device 10 once indwelled in the aneurysm can be aspirated and held again and moved to another position, and the embolic device 10 can be indwelled at an appropriate position according to a state of the aneurysm.

<Delivery System>

The configuration of the delivery system 200 according to the present embodiment will be described. As shown in FIG. 1, the delivery system 200 according to the present embodiment includes, in addition to the medical instrument set 100, the aspiration and ejection unit 30 that aspirates a fluid by setting a pressure inside the aspiration lumen 22 provided in the delivery pusher 20 to be negative and ejects the fluid by setting the pressure inside the aspiration lumen 22 to be positive.

<Aspiration and Ejection Unit>

The aspiration and ejection unit 30 is provided with a mechanism for setting the pressure inside the aspiration lumen 22 of the delivery pusher 20 to be positive or negative. As the aspiration and ejection unit 30, for example, a syringe as shown in FIG. 1 may be used. The syringe causes the pressure inside the aspiration lumen 22 to be positive by pushing the plunger in an ejection direction to eject the fluid, and causes the pressure inside the aspiration lumen 22 to be negative by pulling the plunger in an aspiration direction to aspirate the fluid.

The aspiration and ejection unit 30 is not limited to the syringe as in the present embodiment as long as the aspiration and ejection unit 30 is a mechanism capable of generating an aspirating force sufficient to aspirate and hold the embolic device 10 by aspirating the fluid from at least an opening of the aspiration lumen 22 of the delivery pusher 20, and an electric aspiration device such as an aspiration pump can be used. When the aspiration and ejection unit 30 is a pump, the embolic device delivery medical system 300 may include an operation unit that performs switching between a fluid aspiration operation and a fluid ejection operation of the pump and adjusts a ventilation amount of the fluid, and a control unit that performs ventilation control of the fluid based on an operation amount by the operation unit.

Further, the aspiration and ejection unit 30 preferably has both a fluid aspiration function and a fluid ejection function, and may have at least only the aspiration function. In this case, the aspiration and ejection unit 30 functions as an “aspiration unit” that only performs the fluid aspiration operation. The “gas” as the fluid aspirated or ejected by the aspiration and ejection unit 30 is not particularly limited as long as it is a gas (air, oxygen, or the like) that does not adversely affect the living body in the living body lumen, and physiological saline is preferable.

In this way, the delivery system 200 according to the present embodiment includes the medical instrument set 100 and the aspiration and ejection unit 30 that aspirates or ejects the fluid from the aspiration lumen 22 of the delivery pusher 20.

Therefore, in the delivery system 200, the embolic device 10 once indwelled in the aneurysm by aspirating the fluid by the aspiration and ejection unit 30 can be again aspirated and held by the delivery pusher 20 and moved to another position. In addition, in the delivery system 200, the indwelling position of the embolic device 10 can be finely adjusted by ejecting the fluid from the aspiration lumen 22 when the delivery pusher 20 pushes out the embolic device 10.

<Embolic device Delivery Medical System>

The configuration of the embolic device delivery medical system 300 according to the present embodiment will be described. As shown in FIG. 2, the embolic device delivery medical system 300 provided with the delivery system 200 according to the present embodiment includes, in addition to the delivery system 200 shown in FIG. 1, the delivery catheter 40 for delivering the embolic device 10 into the aneurysm, an insertion assisting member 50 that delivers the delivery catheter 40 into the living body lumen, and an embolic device filled catheter set 60 that loads the embolic device 10 into the delivery catheter 40. As will be described later, the insertion assisting member 50 may be omitted.

<Delivery Catheter>

The delivery catheter 40 is, for example, a catheter formed with the hole (through hole or lumen) extending from the distal end side to the proximal end side along the axial direction, and functions as an introduction path for delivering the embolic device 10 into the aneurysm. The embolic device 10 is loaded into the lumen of the delivery catheter 40 by the embolic device filled catheter set 60. A main body 41 of the delivery catheter 40 can be penetrated by a main body of the insertion assisting member 50 over a total length of the main body 41. Therefore, a length of the main body 41 in the axial direction is at least shorter than that of a main body of the insertion assisting member 50.

A constituent material from which the main body 41 may be fabricated is not particularly limited as long as it is a flexible material. As examples of the constituent material of the main body 41, a resin material such as a polymer material including polyolefin (for example, polyethylene, polypropylene, polybutene, an ethylene-propylene copolymer, an ethylene-vinyl acetate copolymer, an ionomer, or a mixture of two or more types thereof), a polyolefin elastomer, crosslinked polyolefin, polyvinyl chloride, polyamide, a polyamide elastomer, polyester, polyester elastomer, polyurethane, polyurethane elastomer, a fluorous resin, polycarbonate, polystyrene, polyacetal, polyimide, polyetherimide, and aromatic polyether ketone, or a mixture thereof can be suitably used.

The delivery catheter 40 includes a hub 42 interlocked to a proximal end side of the main body 41, and a flexible tube 43 having one end connected to a proximal end side of the hub 42 and the other end connected to a three-way stopcock 44.

The hub 42 is an intermediate member that includes a lumen that allows the main body 41 and the tube 43 to communicate with each other, and allows a fluid (such as physiological saline for priming) flowing from the three-way stopcock 44 to flow to the main body 41 via the tube 43. The insertion assisting member 50 is inserted through the hub 42. A constituent material from which the hub 42 may be fabricated may be the same as the material exemplified as the constituent material of the hub 23 described above.

A hemostasis valve (not shown) is attached into a proximal end side of the hub 42. As the hemostasis valve, for example, a substantially elliptical film-shaped (disk-shaped) valve body made of silicone rubber, latex rubber, butyl rubber, or isoprene rubber which is an elastic member may be used.

The one end of the tube 43 is interlocked to the proximal end side of the hub 42, and the other end is connected to a port 45 of the three-way stopcock 44. The tube 43 is a conduit through which a liquid such as physiological saline flowing out from a priming syringe (not shown) interlocked to the port 45 flows. A constituent material of the tube 43 may be the same as the material exemplified as the constituent material of the tube 24 described above.

The three-way stopcock 44 communicates with the lumen of the hub 42 and the lumen of the main body 41 via the tube 43. In addition to a proximal end side of the tube 43, a priming syringe for priming the lumen of the main body 41 and a liquid injection syringe for injecting a contrast agent, a drug, or the like can be connected to the port 45 of the three-way stopcock 44.

<Insertion Assisting Member>

The insertion assisting member 50 is formed with a guide wire lumen 51 that extends from the distal end side of the insertion assisting member 50 to the proximal end side of the insertion assisting member 50 along the axial direction, and is an assisting tool that assists insertion when the delivery catheter 40 is to be delivered into the aneurysm along a guide wire GW inserted into or positioned in the living body lumen in advance. The insertion assisting member 50 is inserted into and assembled to the delivery catheter 40 in order to prevent the delivery catheter 40 from being broken when the delivery catheter 40 is inserted into the living body lumen. The guide wire lumen 51 has an inner diameter smaller than that of a lumen 20a of the delivery catheter 40. Therefore, when the delivery catheter 40 is delivered into the aneurysm, an axial deviation of the delivery catheter 40 with respect to the guide wire GW can be reduced, leading to easier delivery.

A constituent material from which the insertion assisting member 50 may be fabricated is not particularly limited as long as it is harder and more flexible than the delivery catheter 40. As examples of the constituent material of the insertion assisting member 50, a resin material including polyolefin such as polyethylene and polypropylene, polyester such as polyamide and polyethylene terephthalate, a fluorous resin such as ETFE, polyetheretherketone PEEK, polyimide, and a metal material such as a shape memory alloy, stainless steel, tantalum, titanium, platinum, gold, and tungsten can be suitably used.

In the embolic device delivery medical system 300, the insertion assisting member 50 may be omitted. In this case, the lumen 20a of the delivery catheter 40 also serves as the guide wire lumen 51.

<Embolic Device Filled Catheter Set>

The embolic device filled catheter set 60 is for loading the embolic device 10 into the lumen of the delivery catheter 40. The embolic device filled catheter set 60 includes an embolic device filled catheter 61 that is pre-filled with the embolic device 10, and an elongated push-out pusher 66 that is inserted into the embolic device filled catheter 61 in order to push the embolic device 10 accommodated in the embolic device filled catheter 61 into the lumen 20a of the delivery catheter 40.

The embolic device filled catheter 61 includes a filling catheter main body 61a filled with the embolic device 10, a hub 62 to which a proximal end side of the filling catheter main body 61a is connected, and a flexible tube 63 having one end connected to a proximal end side of the hub 62 and the other end connected to a port 65 of a three-way stopcock 64.

The filling catheter main body 61a is a tubular member formed with a hole (through hole or lumen) extending from the distal end side to the proximal end side along the axial direction, and can accommodate the embolic device 10 inserted from the distal end side. A length of the filling catheter main body 61a in an axially extending direction is appropriately defined, and it is sufficient that the filling catheter main body 61a has a length at least capable of accommodating the embolic device 10. The filling catheter main body 61a is provided mainly in a state in which the embolic device 10 is loaded in advance, and the embolic device 10 to be accommodated in the filling catheter main body 61a may be accommodated in the filling catheter main body 61a by the surgeon or the like gripping the embolic device 10.

The embolic device 10 positioned in the filling catheter main body 61a embolic device is pushed out by the push-out pusher 66 in a state in which the filling catheter main body 61a is inserted into the hub 42 of the delivery catheter 40, thereby resulting in the embolic device 10 being loaded into the lumen of the main body 41 of the delivery catheter 40. Further, the filling catheter main body 61a can be employed by using the delivery pusher 20 to push-out the embolic device 10 in a state in which the filling catheter main body 61a is inserted into or positioned in the delivery catheter 40.

A constituent material from which the filling catheter main body 61a may be fabricated is not particularly limited as long as it is a material having appropriate hardness and flexibility. As examples of the constituent material of the filling catheter main body 61a, a resin material such as a polymer material including polyolefin (for example, polyethylene, polypropylene, polybutene, an ethylene-propylene copolymer, an ethylene-vinyl acetate copolymer, an ionomer, or a mixture of two or more types thereof), a polyolefin elastomer, crosslinked polyolefin, polyvinyl chloride, polyamide, a polyamide elastomer, polyester, polyester elastomer, polyurethane, polyurethane elastomer, a fluorous resin, polycarbonate, polystyrene, polyacetal, polyimide, polyetherimide, and aromatic polyether ketone, or a mixture thereof, and a metal material such as a shape memory alloy, stainless steel, tantalum, titanium, platinum, gold, or tungsten can be suitably used.

The hub 62 is an intermediate member that includes a lumen that allows the filling catheter main body 61a and the tube 63 to communicate with each other, and allows a fluid (such as physiological saline for priming) flowing from the three-way stopcock 64 to flow to the filling catheter main body 61a via the tube 63. The push-out pusher 66 is inserted through the hub 62, and the embolic device 10 accommodated in the filling catheter main body 61a is pushed out. A constituent material from which the hub 62 may be fabricated may be the same as the materials exemplified as the constituent materials of the hubs 23 and 42 described above.

The one end of the tube 63 is interlocked to the proximal end side of the hub 62, and the other end is connected to the port 65 of the three-way stopcock 64. The tube 63 is a conduit through which a liquid such as physiological saline flowing out from a priming syringe (not shown) interlocked to the port 65 flows. A constituent material from which the tube 63 may be fabricated may be the same as the materials exemplified as the constituent materials of the tubes 24 and 43 described above.

The three-way stopcock 64 communicates with the lumen of the hub 62 and the lumen of the filling catheter main body 61a via the tube 63. In addition to a proximal end side of the tube 63, a priming syringe for priming the lumen of the filling catheter main body 61a can be connected to the port 65 of the three-way stopcock 64.

The push-out pusher 66 is a rod-shaped member that is positioned in or inserted through the hub 62 for pushing out the embolic device 10 accommodated in the filling catheter main body 61a and loading the embolic device 10 into the lumen of the delivery catheter 40. When the embolic device 10 is to be loaded into the delivery catheter 40, the push-out pusher 66 is inserted from the hub 62 and inserted into the lumen of the filling catheter main body 61a to push out the embolic device 10.

A constituent material from which the push-out pusher 66 may be fabricated is not particularly limited, and is a material having rigidity with which the embolic device 10 can be pushed out. As examples of the constituent material of the push-out pusher 66, polyolefin such as polyethylene and polypropylene, polyamide, polyester such as polyethylene terephthalate, a fluorous resin such as ETFE, polyetheretherketone (PEEK), polyimide, and a metal such as a shape memory alloy, stainless steel, tantalum, titanium, platinum, gold, and tungsten can be suitably used.

In this way, the embolic device delivery medical system 300 according to the present embodiment includes the delivery system 200, the delivery catheter 40 for delivering the embolic device 10 into the aneurysm, the insertion assisting member 50 assembled to the delivery catheter 40 for assisting delivery of the delivery catheter 40 into the aneurysm along the guide wire GW inserted into the living body lumen in advance, and the embolic device filled catheter set 60 that loads the embolic device 10 into the delivery catheter 40, and the embolic device 10 is loaded into the lumen of the delivery catheter 40 by operating the push-out pusher 66 in a state in which the embolic device 10 is accommodated in the filling catheter main body 61a of the embolic device filled catheter set 60.

Accordingly, even when the embolic device 10 is relatively elongated, the embolic device 10 can be smoothly loaded into the delivery catheter 40. Further, when the delivery catheter 40 is to be inserted into the living body lumen, the delivery catheter 40 can be smoothly inserted by performing the insertion in a state in which the insertion assisting member 50 is assembled (i.e., in a state in which the insertion assisting member 50 is positioned in and axially overlaps the delivery catheter 40.

[Operation]

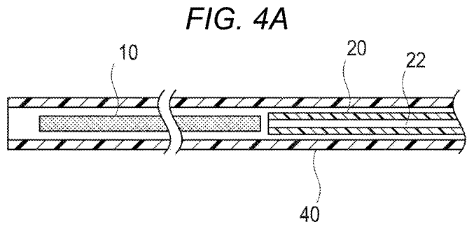

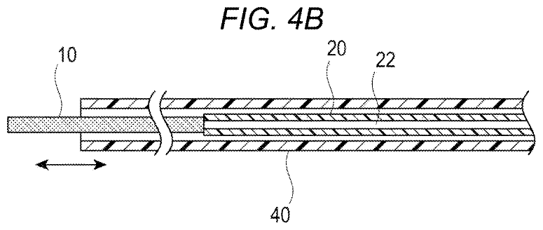

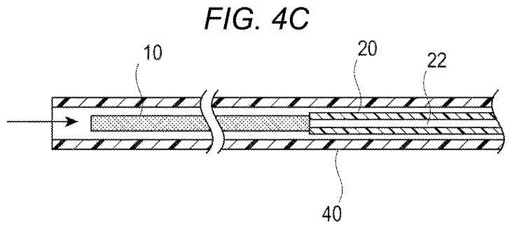

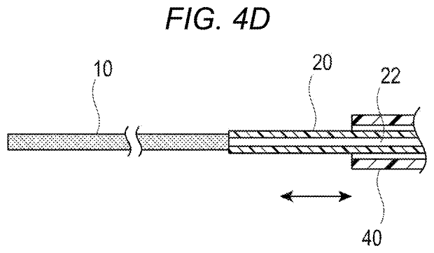

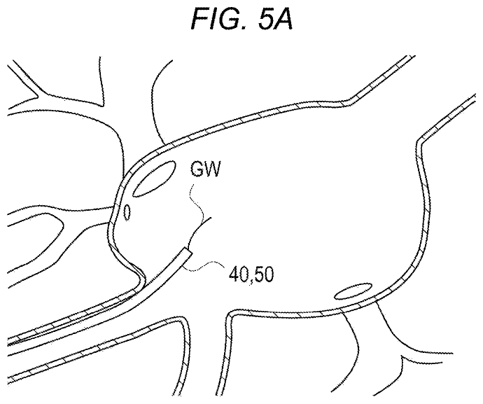

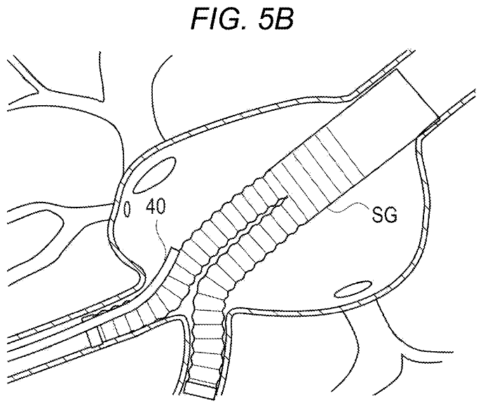

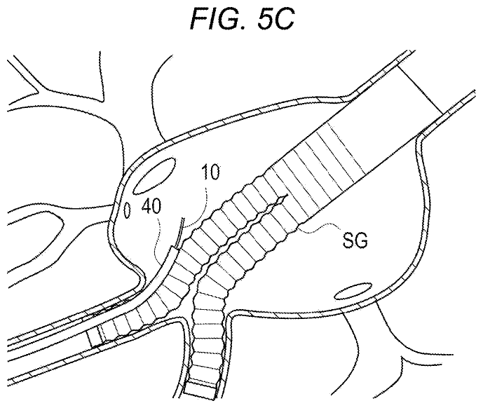

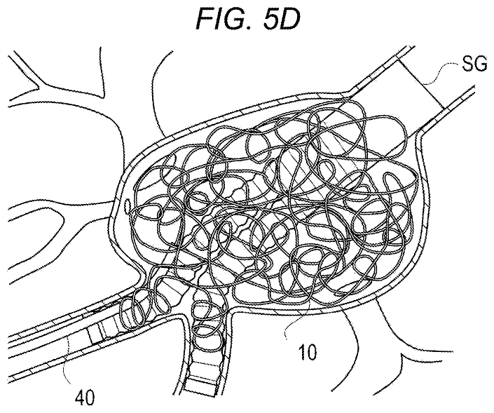

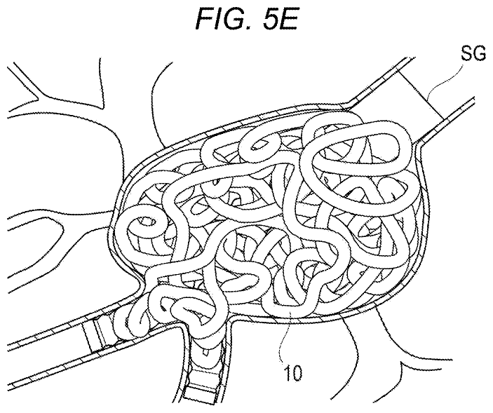

Next, an operation of the embolic device delivery medical system 300 according to the present embodiment will be described. FIGS. 4A to 4D show an example of a conveyance operation (first operation) of the embolic device 10 by the delivery pusher 20 in the embolic device delivery medical system 300, and FIGS. 5A to 5E show an indwelling operation (second operation) of the embolic device 10 along a main procedure step of the endoleak embolization for the stent graft interpolation of the abdominal aortic aneurysm.

<First Operation: Conveyance Operation>

First, the conveyance operation of the delivery pusher 20 in the embolic device delivery medical system 300 according to the present embodiment will be described with reference to FIGS. 4A to 4D. Each operation here is an operation example that is appropriately performed at a time of a conveyance step in an “embolic device indwelling operation” to be described later.

FIG. 4A shows a state in which the distal end side of the main body 21 of the delivery pusher 20 inserted from the proximal end side of the delivery catheter 40 reaches the vicinity of the proximal end side of the embolic device 10 loaded in the lumen of the delivery catheter 40. In this state, the delivery pusher 20 is not subjected to the fluid aspiration operation and fluid ejection control.

As shown in FIG. 4B, the embolic device 10 pushed out by the delivery pusher 20 from the state of FIG. 4A is indwelled in the aneurysm while gradually moving toward the distal end side in the lumen of the delivery catheter 40 based on an operation of the surgeon. At a time of a pushing-out operation by the delivery pusher 20 shown in FIG. 4B, the embolic device 10 may be conveyed in a state in which the embolic device 10 is aspirated and held on the distal end side of the delivery pusher 20 in a state in which the fluid aspiration operation by the aspiration and ejection unit 30 is performed, or may be conveyed in a state in which the fluid aspiration operation by the aspiration and ejection unit 30 is not performed.

When the embolic device 10 conveyed into the aneurysm is not indwelled at an indwelling position desired by the surgeon, as shown in FIG. 4C, the surgeon can perform the fluid aspiration operation with the aspiration and ejection unit 30, and can pull back the embolic device 10 in the aneurysm into the lumen of the delivery catheter 40 with the embolic device 10 being aspirated and held at the distal end of the delivery pusher 20 while operating the delivery pusher 20.

Further, as shown in FIG. 4D, when the surgeon performs a predetermined operation on the delivery pusher 20 in a state in which the aspiration and ejection unit 30 is subjected to the fluid aspiration operation, the embolic device 10 is released from the delivery catheter 40 (i.e., moved out of the delivery catheter 40 so that the embolic device 10 is distal of the distal end of the delivery catheter as shown in FIG. 4D) while being aspirated and held, and can be moved in the aneurysm. Accordingly, the surgeon can convey and indwell the embolic device 10 aspirated and held by the delivery pusher 20 to a desired indwelling position in the aneurysm.

When the fluid aspiration operation of the aspiration and ejection unit 30 is stopped in a state in which the embolic device 10 is aspirated and held by the delivery pusher 20 shown in FIGS. 4C and 4D, the aspirated-and-held state of the embolic device 10 by the delivery pusher 20 is released. That is, when the fluid aspiration operation by the aspiration and ejection unit 30 is stopped at a position at which the embolic device 10 is desired to be indwelled, the embolic device 10 can be indwelled at the position.

Further, when the indwelling position of the embolic device 10 is desired to be a position further deeper than a current aspiration-holding position, the aspiration and ejection unit 30 can perform the fluid ejection operation, and eject the fluid from the aspiration lumen 22 of the delivery pusher 20 to blow off the embolic device 10 to a desired position, thereby indwelling the embolic device 10.

As described above, in the embolic device delivery medical system 300, by operating the delivery pusher 20 while selectively using fluid aspiration control and fluid ejection control of the aspiration and ejection unit 30, the indwelling position of the embolic device 10 can be controlled, and the indwelling position can be readjusted even for the embolic device 10 indwelled once.

<Second Operation: Embolic Device Indwelling Operation>

Next, with reference to FIGS. 5A to 5E, an operation example of the embolic device delivery medical system 300 according to the present embodiment will be described along a flow of endoleak embolization for the stent graft interpolation of the abdominal aortic aneurysm.

<Step 1: Preparation Step>

First, in a state in which the insertion assisting member 50 is inserted into or positioned in the delivery catheter 40, the surgeon percutaneously inserts the main body 41 into the living body lumen, and then removes the insertion assisting member 50. FIG. 5A shows a state in which the distal end of the delivery catheter 40 to which the insertion assisting member 50 is assembled is delivered into the aneurysm along the guide wire GW. As shown in FIG. 5A, when the delivery catheter 40 is inserted into the aneurysm, the delivery catheter 40 is inserted in a state in which the insertion assisting member 50 is assembled (i.e., the delivery catheter 40 is inserted into the aneurysm while the insertion assisting member 50 is located in, and is in axially overlapping relation to, the delivery catheter 40).

<Step 2: Stent Graft Expansion and Indwelling Step>

Next, as shown in FIG. 5B, the surgeon inserts, from the delivery catheter 40 into the living body lumen, a catheter (stent graft device) into which a stent graft SG is compressed and inserted, moves the catheter (stent graft device) to an aneurysm target lesion using the guide wire GW, and expands and indwells the stent graft SG from the catheter at the target lesion. In the expansion and indwelling step of the stent graft SG, first, a main body portion is expanded and indwelled, limb portions are attached to bifurcated portions of the main body portion, respectively, and the process ends.

<Step 3: Loading Step>

Next, the surgeon loads the embolic device 10 into the delivery catheter 40 using the embolic device filled catheter set 60. As a method of loading the embolic device 10 into the delivery catheter 40, the embolic device filled catheter set 60 in which the filling catheter main body 61a is filled with the embolic device 10 may be prepared, and in a state in which the filling catheter main body 61a is inserted into or positioned in the delivery catheter 40, the push-out pusher 66 may be inserted into the filling catheter main body 61a via the hub 62 to push out the embolic device 10 and load the embolic device 10 into the lumen of the main body 41. When the embolic device 10 is directly pushed into the aneurysm from the filling catheter main body 61a, the loading step is skipped. In this case, by inserting the filling catheter main body 61a loaded with the embolic device 10 into the delivery catheter 40, the embolic device 10 is loaded (accommodated) in the delivery catheter 40 as in the loading step.

<Step 4: Conveyance Step>

Next, the surgeon inserts the main body 21 of the delivery pusher 20 into the lumen of the delivery catheter 40 loaded with the embolic device 10 via the hub 42, and pushes the embolic device 10 into the aneurysm. In a conveyance step, the surgeon conveys the embolic device 10 to a desired indwelling position in the aneurysm while performing a predetermined operation on the delivery pusher 20 and the aspiration and ejection unit 30. At this time, the surgeon conveys the embolic device 10 while appropriately using the conveyance operation as shown in FIGS. 4B to 4D. The surgeon repeats the loading step and the conveyance step such that an amount of the embolic device 10 suitable for the patient is indwelled in the aneurysm while confirming a size of the aneurysm and an indwelling amount of the embolic device 10. Further, as described in the loading step, when the embolic device 10 is directly pushed out from the filling catheter main body 61a into the aneurysm to be indwelled, the embolic device 10 can be pushed out by using the delivery pusher 20 instead of the push-out pusher 66.

<Step 5: Removal Step>

Thereafter, as shown in FIG. 5D, when the embolic device 10 is indwelled in the aneurysm, the surgeon removes the delivery catheter 40.

<Step 6: Expansion and Closing Step>

The embolic device 10 indwelled in the aneurysm comes into contact with a liquid such as blood in the aneurysm and gradually swells, and as shown in FIG. 5E, the completely expanded embolic device 10 fills a space between an inner surface of the aneurysm and an outer surface of the stent graft SG, thereby closing the aneurysm. Accordingly, the aneurysm is prevented from rupturing.

As described above, the medical instrument set 100 according to the present embodiment includes the embolic device 10 insertable into the delivery catheter 40, and configured to, when the embolic device 10 is indwelled in the aneurysm, absorb a liquid in the aneurysm and expand, and the elongated delivery pusher 20 insertable into the delivery catheter 40 and configured to push the embolic device 10 out of the delivery catheter 40 into the aneurysm. The delivery pusher 20 includes the elongated main body 21, and the aspiration lumen 22 that is formed to penetrate the main body 21 in the axial direction from a distal end to a proximal end of the main body 21 and causes the delivery pusher 20 to aspirate and hold the embolic device 10.

The embolic device delivery medical system 300 according to the present embodiment includes the delivery system 200 according to the present embodiment, the delivery catheter 40 for delivering the embolic device 10 into the aneurysm, and the embolic device filled catheter set 60 including the embolic device filled catheter 61 that includes a lumen for filling and is insertable into the lumen of the delivery catheter 40 in a state in which the lumen for filling is filled with the embolic device 10 and the elongated push-out pusher 66 that pushes out the embolic device 10 from the lumen for filling.

With such a configuration, in the medical instrument set 100, the embolic device 10 once indwelled in the aneurysm can be aspirated and held again by the delivery pusher 20. Therefore, the indwelling position of the embolic device 10 can be moved to another position, and the embolic device 10 can be indwelled at an appropriate position according to the state of the aneurysm. Therefore, even when the embolic device 10 enters slight gaps generated at connection portions between the bifurcated portions and the limb portions of the main body portion in the stent graft SG used in the stent graft interpolation, or a side branch blood vessel branched from the aneurysm, the embolic device 10 can be moved to an appropriate indwelling position by being aspirated and held. Therefore, it is possible to prevent a problem including occurrence of an endoleak caused by the embolic device 10.

When the embolic device 10 is pushed out by the delivery pusher 20, the indwelling position of the embolic device 10 can be finely adjusted by ejecting the fluid from the aspiration lumen 22. Further, even the embolic device 10 is relatively elongated, the embolic device 10 can be smoothly loaded into the delivery catheter 40 by using the embolic device filled catheter set 60. The embolic device filled catheter 61 included in the embolic device filled catheter set 60 can be inserted into the delivery catheter 40 in a state in which the embolic device 10 is filled in advance, and the embolic device 10 can be used without being transferred into the delivery catheter 40.

### Other Embodiments

The invention is not limited to the above-described embodiment, and for example, as to be described below, the invention can be appropriately modified and implemented in accordance with use environments or the like. In addition, the following modifications may be implemented in any combination without departing from the scope of the invention.

### First Modification

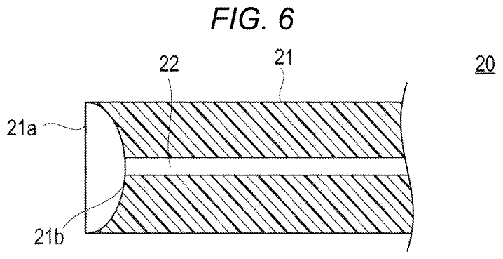

As shown in FIG. 6, in a first modification, the delivery pusher 20 according to the first modification has a configuration in which an engagement concave portion 21b curved and recessed from a distal end surface 21a of the main body 21 toward the proximal end side is formed in the distal end surface 21a. Specifically, the engagement concave portion 21b has a curved surface (concave curved surface) whose inner surface is curved in an arc shape from the distal end side toward the proximal end side in a cross section parallel to the axial direction of the main body 21.

According to the delivery pusher 20 of the first modification, by forming the engagement concave portion 21b in the distal end surface 21a of the main body 21, an impact on the embolic device 10 at a time of aspirating is dispersed as compared to a state in which the distal end surface 21a is flat, and damage to the embolic device 10 can be prevented. Further, since the proximal end side of the embolic device 10 enters a concave portion during a fluid aspiration operation, the embolic device 10 can be aspirated and held more easily than when the distal end surface 21a of the main body 21 is a flat surface.

### Second Modification

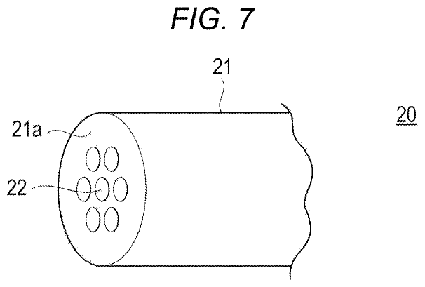

As shown in FIG. 7, a second modification has a configuration in which a plurality of aspiration lumens 22 are formed in the main body 21 of the delivery pusher 20. FIG. 7 shows a configuration example in which seven aspiration lumens 22 are formed. The number of aspiration lumens 22 is not particularly limited, and may be appropriately set as long as the hole diameter (diameter similar to the diameter r1 shown in FIG. 3B) is smaller than at least the width dimension (dimension similar to the dimension r2 shown in FIG. 3B) (diameter) of the embolic device 10 to be used. In addition, the plurality of aspiration lumens 22 may communicate from the distal end side to the proximal end side, or may join together in a middle of a portion from a distal end portion to a proximal end portion to become one lumen.

According to the delivery pusher 20 of the second modification, by forming the plurality of aspiration lumens 22, a plurality of aspiration portions are provided with respect to a proximal end portion of the embolic device 10. Therefore, as compared to a case in which the aspiration lumen 22 is provided alone, the embolic device 10 can be aspirated and held easily even with a small aspirating force.

### Third Modification

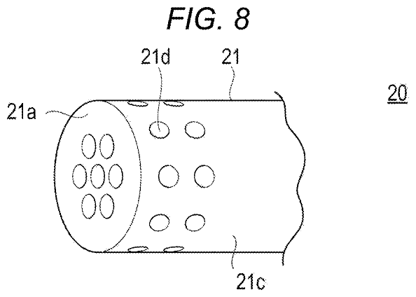

As shown in FIG. 8, the delivery pusher 20 according to a third modification has a configuration in which communication holes 21d communicating with the aspiration lumen 22 are formed in an outer peripheral surface 21c on a distal end side of the main body 21 of the delivery pusher 20. In FIG. 8, a plurality of communication holes 21d are formed in two rows aligned in the axial direction along the outer peripheral surface (outer circumferential surface) 21c of the main body 21 at predetermined intervals. The number of the communication holes 21d and the intervals therebetween are not particularly limited, and it is sufficient that a hole size of each of the communication holes 21d is narrower or smaller than at least the width dimension (diameter) of the embolic device 10 to be used.

According to the delivery pusher 20 of the third modification, since the embolic device 10 can be aspirated and held by both ventilation holes of the aspiration lumen 22 formed in the distal end surface 21a serving as a distal end portion of the main body 21 and the communication holes 21d formed in the outer peripheral surface 21c, the embolic device 10 separated from the main body 21 of the delivery pusher 20, such as the embolic device 10 completely protruding from the delivery catheter 40 and the embolic device 10 that is already indwelled, can be easily aspirated and held.

The detailed description above describes embodiments of a medical instrument set, delivery system, and embolic device delivery medical system representing examples of the inventive medical instrument set, delivery system, and embolic device delivery medical system disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical instrument set comprising:
an embolic device insertable into a delivery catheter, indwellable in an aneurysm and configured to, when the embolic device is indwelled in the aneurysm, absorb liquid in the aneurysm and expand;
a delivery pusher insertable into the delivery catheter and configured to push the embolic device out of the delivery catheter into the aneurysm; and
the delivery pusher including an elongated main body, and an aspiration lumen that extends through the elongated main body in an axial direction from a distal end of the elongated main body to a proximal end of the elongated main body and that allows aspiration to be applied to the embolic device at the distal end of the delivery pusher to hold the embolic device.

2. The medical instrument set according to claim 1, wherein the delivery pusher includes a distal end surface at the distal end of the delivery pusher, the distal end surface of the delivery pusher is a concave curved surface curved in an arc shape from a distal end side of the delivery pusher toward a proximal end side of the delivery pusher in a cross section parallel to an axial direction of the main body.

3. The medical instrument set according to claim 1, wherein the aspiration lumen is a first aspiration lumen, the delivery pusher a plurality of additional aspiration lumens extending through the elongated main body in the axial direction from the distal end of the elongated main body to the proximal end of the elongated main body.

4. The medical instrument set according to claim 1, wherein the delivery pusher includes a communication hole extending from an outer circumferential surface of the main body of the delivery pusher to the aspiration lumen so that the aspiration lumen communicates with the outer circumferential surface of the main body.

5. The medical instrument set according to claim 1, wherein the delivery pusher incudes a distal end surface at the distal end of the delivery pusher, the embolic device possessing a width dimension, the aspiration lumen possessing an inner diameter that, at the distal end surface of the delivery pusher, is less than width dimension of the embolic device.

6. An embolic device delivery medical system comprising:
a tubular delivery catheter that includes a distal portion terminating at a distal end, the tubular delivery catheter also including a proximal end and a lumen extending between the distal end and the proximal end of the tubular delivery catheter, the tubular delivery catheter being configured to have its distal portion introduced into a living body and positioned in an aneurysm in the living body;
an embolic device-filled catheter set that includes an embolic device in combination with an embolic device catheter, the embolic device catheter including a distal end, a proximal end and a lumen extending between the distal end and the proximal end, the embolic device being positioned in the lumen of the embolic device catheter, the embolic device being removable from the lumen of the embolic device catheter and indwellable in an aneurysm, the embolic device being configured to absorb liquid in the aneurysm when the embolic device is indwelled in the aneurysm and to expand upon absorbing the liquid, the embolic device catheter being configured to be inserted into the lumen in the tubular delivery catheter when the embolic device is positioned in the lumen in the embolic device catheter;
a delivery pusher that includes an elongated main body and an aspiration lumen, the elongated main body of the delivery pusher being insertable into the delivery catheter and configured to push the embolic device out of the delivery catheter when the distal portion of the tubular delivery catheter is positioned in the aneurysm in the living body so that the embolic device is located in the aneurysm; and
the aspiration lumen extending through the elongated main body of the delivery pusher in an axial direction from a distal end of the elongated main body to a proximal end of the elongated main body to permit aspiration to be applied to the embolic device when the embolic device is located at the distal end of the elongated main body to hold the embolic device at the distal end of the elongated main body of the delivery pusher.

7. The embolic device delivery medical system according to claim 6, further comprising: an insertion assisting member positionable in the lumen in the delivery catheter to assist movement of the delivery catheter in the living body and delivery of the delivery catheter into the aneurysm.

8. The embolic device delivery medical system according to claim 6, further comprising a pusher that includes a rod-shaped member positionable in the lumen of the embolic device catheter when the embolic device is in the lumen of the embolic device catheter and the embolic device catheter is positioned in the lumen in the tubular delivery catheter to push-out the embolic device from the lumen of the embolic device catheter and into the lumen in the delivery catheter.

9. The embolic device delivery medical system according to claim 6, further comprising an aspiration and ejection unit that is in communication with the lumen in the delivery pusher and that is configured to be operated to: i) apply aspiration to the distal end of the elongated main body to hold the embolic device against the distal end of the elongated main body; and ii) apply ejection to the distal end of the elongated main body to eject the embolic device away from the distal end of the elongated main body.

10. The embolic device delivery medical system according to claim 6, further comprising a syringe that is in communication with the lumen in the delivery pusher by way of tubing.

11. The embolic device delivery medical system according to claim 6, wherein the delivery pusher includes a distal end surface at the distal end of the delivery pusher, the distal end surface of the delivery pusher is a concave curved surface curved in an arc shape from a distal end side of the delivery pusher toward a proximal end side of the delivery pusher in a cross section parallel to an axial direction of the main body.

12. The embolic device delivery medical system according to claim 6, wherein the aspiration lumen is a first aspiration lumen, the delivery pusher a plurality of additional aspiration lumens extending through the elongated main body in the axial direction from the distal end of the elongated main body to the proximal end of the elongated main body.

13. The embolic device delivery medical system according to claim 6, wherein the delivery pusher includes a communication hole extending from an outer circumferential surface of the main body of the delivery pusher to the aspiration lumen so that the aspiration lumen communicates with the outer circumferential surface of the main body.

14. The embolic device delivery medical system according to claim 6, wherein the delivery pusher incudes a distal end surface at the distal end of the delivery pusher, the embolic device possessing a width dimension, the aspiration lumen possessing an inner diameter that, at the distal end surface of the delivery pusher, is less than width dimension of the embolic device.

15. The embolic device delivery medical system according to claim 6, further comprising an elongated insertion assisting member positionable in the lumen in the delivery catheter to assist movement of the delivery catheter in the living body and delivery of the delivery catheter into the aneurysm, the elongated insertion assisting member including a distal end, a proximal end and a guide wire lumen extending throughout the elongated insertion assisting member from the distal end of the elongated insertion assisting member to the proximal end of the elongated insertion assisting member.

16. A method comprising:
introducing a distal portion of a delivery catheter into a living body and positioning the distal end of the delivery catheter in an aneurysm in the living body, the delivery catheter including a distal end, a proximal end and a lumen extending throughout the delivery catheter from the distal end of the delivery catheter to the proximal end of the delivery catheter;
introducing a distal end of a delivery pusher into the delivery catheter by way of the proximal end of the delivery catheter, the introducing of the distal end of the delivery pusher into delivery catheter by way of the proximal end of the delivery catheter including introducing the distal end of the delivery pusher into the delivery catheter while an embolic device is located inside the delivery catheter; moving the delivery pusher in a forward direction to push the embolic device out of the delivery catheter so that the embolic device is located outside the delivery catheter and in the aneurysm;
applying aspiration to the lumen in the delivery pusher to hold the embolic device that is located in the aneurysm against a distal end of the delivery pusher;
moving the embolic device in the aneurysm while the embolic device is held against the distal end of the delivery pusher by the aspiration; \
stopping the applying of the aspiration to the lumen in the delivery pusher to release the embolic device in the aneurysm; and
moving the delivery pusher in a rearward direction while the embolic device remains in the aneurysm.

17. The method according to claim 16, further comprising introducing an embolic device catheter into the lumen in the delivery catheter before introducing a distal end of a delivery pusher into the delivery catheter by way of the proximal end of the delivery catheter, the embolic device catheter including a lumen extending throughout the embolic device catheter, the embolic device being positioned in the lumen in the embolic device catheter during the introducing of the embolic device catheter into the lumen in the delivery catheter, the introducing of the distal end of the delivery pusher into the delivery catheter by way of the proximal end of the delivery catheter including introducing the distal end of the delivery pusher into the lumen in the embolic device catheter.

18. The method according to claim 17, further comprising: i) introducing a pusher into the lumen in embolic device catheter after the introducing of the embolic device catheter into the lumen in the delivery catheter; and ii) using the pusher to push the embolic device out of the lumen in the embolic device catheter and into the lumen in the delivery catheter.

19. The method according to claim 16, further comprising delivering an ejection fluid to the lumen in the delivery pusher to eject the embolic device away from the distal end of the delivery pusher, the delivering of the ejection fluid to the lumen in the delivery pusher occurring after the applying of the aspiration to the lumen in the delivery pusher.
